(19) United States
(12) Patent Application Publication
(10) Pub. No.: US 2015/0250377 A1
(43) Pub. Date: Sep. 10, 2015

(54) ENDOSCOPE SYSTEM

(71) Applicant: OLYMPUS CORPORATION, Tokyo (JP)

(72) Inventor: Tomoyuki IIZUKA, Tokyo (JP)

(73) Assignee: OLYMPUS CORPORATION, Tokyo (JP)

(21) Appl. No.: 14/713,642

(22) Filed: May 15, 2015

Related U.S. Application Data
(63) Continuation of application No. PCT/JP2014/060326, filed on Apr. 9, 2014.

(30) Foreign Application Priority Data
May 22, 2013 (JP) 2013-108137

(51) Int. Cl. A61B 1/00; A61B 1/05

(52) U.S. Cl. CPC A61B 1/00009; A61B 1/00045; A61B 1/05; A61B 1/00087; A61B 1/0002; A61B 1/00006; A61B 1/00183

(57) ABSTRACT

An endoscope system of the present invention is provided with an image pickup device, a treatment instrument raising base, a signal processing section and a monitor.

### CROSS REFERENCE TO RELATED APPLICATION

This application is a continuation application of PCT/JP2014/060326 filed on Apr. 9, 2014 and claims benefit of Japanese Application No. 2013-108137 filed in Japan on May 22, 2013, the entire contents of which are incorporated herein by this reference.

### BACKGROUND OF THE INVENTION

1. Field of the Invention

The present invention relates to an endoscope system equipped with an endoscope.

2. Description of the Related Art

In recent years, endoscopes are widely used in a medical field and an industrial field. Endoscopes used in the medical field can observe an inside of a body cavity by inserting an elongated insertion portion into the body cavity of a subject. Furthermore, endoscopes used in the medical field can perform various types of treatment on a site to be observed using a treatment instrument inserted into a channel provided for the endoscope as required.

On the other hand, endoscopes used in the industrial field can observe damage or corrosion on a site to be observed of an object or inspect various types of treatment by inserting an elongated insertion portion of the endoscopes into the object such as a jet engine or pipes at factories.

In addition, as endoscopes used in the medical field and the industrial field, front-view type endoscopes are well known which are provided with an objective lens on a distal end surface of a distal end portion located at a distal end (hereinafter simply referred to as “distal end”) in an insertion direction of an insertion portion to observe a forward direction in the insertion direction (hereinafter simply referred to as “forward direction”) using the objective lens. Moreover, as endoscopes used in the medical field and the industrial field, side-view type endoscopes are well known which are provided with an objective lens on part of an outer circumferential face of a distal end portion to observe a side direction with respect to the insertion direction using the objective lens.

Here, when the endoscope used is an electronic endoscope, a subject (object) is observed using, for example, an image pickup unit provided in the distal end portion of the insertion portion. Note that the image pickup unit is constructed of one or a plurality of lenses and provided with an objective optical system including the aforementioned objective lens. The image pickup unit is also provided with an image pickup device having a light-receiving surface on which an image of a site to be observed is formed via the objective optical system.

An observed image captured by the image pickup unit is transmitted to a processor to which a connector is connected via a signal cable, imaged in the processor and then displayed on a display section. Note that the signal cable is inserted through an insertion portion of the endoscope, an operation section connected to a proximal end of the insertion portion in the insertion direction (hereinafter simply referred to as “proximal end”), a universal cord extending from the operation section, and a connector provided at an extending end of the universal cord.

Japanese Patent Application Laid-Open Publication No. 2000-279380 discloses a configuration of an endoscope system provided with an electronic endoscope that can select a desired visual field range from among images displayed on a display section through operation from an operation section, that is, observation visual fields of an objective lens and show an enlarged view of the selected visual field range on the display section and a peripheral apparatus to which the electronic endoscope is connected.

Endoscopes used in the medical field are generally used to observe and treat an affected area located in a digestive tract system, a pancreaticobiliary duct system or the like.

Examples of treatment of the pancreaticobiliary duct system or the like using this endoscope include diagnostic treatment that performs contrast imaging of the bile duct or pancreatic duct using a side-view type endoscope, treatment that collects gallstones located in the common bile duct using a treatment instrument, treatment that resects the gastric mucous layer using a treatment instrument via a front-view type endoscope, hemostasis of a bleeding region or the like.

However, when performing endoscopic therapy on the pancreatic duct, bile duct, hepatic duct or the like using a side-view type endoscope, for example, since the pancreatic duct, bile duct or hepatic duct or the like is a duct having quite a small diameter, it is difficult to directly insert the distal end portion of the insertion portion of the endoscope into these ducts.

For this reason, when performing endoscopic therapy on the pancreatic duct, bile duct, hepatic duct or the like, the distal end portion of the insertion portion of the endoscope is normally inserted into the vicinity of the duodenal papilla. Next, under X-ray fluoroscopy, a guide wire is ejected from an opening of a channel formed at part of the outer circumferential face of the distal end portion and inserted into the aforementioned duct. After that, a technique is used whereby a treatment instrument such as a catheter is selectively inserted into the pancreatic duct, bile duct or hepatic duct using the guide wire as a guide.

A configuration is also well known in which using a treatment instrument raising base, the treatment instrument is inserted into the aforementioned duct via the channel opening Note that the treatment instrument raising base is provided at a position inside the distal end portion facing the channel opening in the channel. Furthermore, the treatment instrument raising base changes an advancing direction of the guide wire or treatment instrument (hereinafter referred to as “treatment instrument” for simplification) inserted into the channel toward the channel opening side by raising.

Note that the treatment instrument raising base is vertically movable by operation from the operation section. On a display section, the treatment instrument raising base or the treatment instrument raised by the treatment instrument raising base are displayed as moving from bottom to top on a screen.

Here, when raising the treatment instrument using the treatment instrument raising base and inserting the treatment instrument into the aforementioned duct, the raising angle of the treatment instrument raising base may need to be increased depending on the position of a site to be observed. For example, when inserting the treatment instrument into the bile duct, for reasons related to the position at which the bile duct extends from the papilla, the greater the raising angle of the treatment instrument raising base, the easier it becomes to insert the treatment instrument into the bile duct.

In addition, when raising a treatment instrument with weak resistance, if the raising angle of the treatment instrument raising base is excessively increased, the treatment instrument may be broken, and so the raising angle of the treatment instrument raising base needs to be decreased.

Moreover, even when a wide angle lens is used for an objective lens, using a configuration of an endoscope system disclosed in Patent Literature 1 to select an image displayed on a display section, that is, a desired visual field range from an observation visual field of an objective lens and display an enlarged view thereof on the display section will facilitate the operation of inserting the treatment instrument into the aforementioned duct.

More specifically, if an enlarged view is displayed such that only a periphery of the raised treatment instrument is located in the center of the display section, this will not only improve observability of the treatment instrument but also facilitate insertion of the treatment instrument into the aforementioned duct.

### SUMMARY OF THE INVENTION

An endoscope system according to an aspect of the present invention includes an image pickup device that has a light-receiving surface on which an image of a site to be observed in a subject is formed via an objective optical system, a treatment instrument guiding member that guides a treatment instrument in an observation visual field of the objective optical system to the site to be observed in the subject, a signal processing section that receives from the image pickup device, an electric signal of an entirety of the observation visual field whose image is formed on the light-receiving surface, extracts the electric signal of any one range of a plurality of visual field ranges set along a guiding direction of the treatment instrument guiding member with respect to the light-receiving surface from the electric signal and images the extracted visual field range corresponding to the electric signal, and a display section that displays an image of the extracted visual field range imaged by the signal processing section.

An endoscope system according to another aspect of the present invention includes an image pickup device that has a light-receiving surface on which an image of a site to be observed in a subject is formed via an objective optical system, a treatment instrument guiding member that guides a treatment instrument in an observation visual field of the objective optical system to the site to be observed in the subject, a signal extraction section that receives from the image pickup device, an electric signal of an entirety of the observation visual field of the objective optical system whose image is formed on the light-receiving surface and extracts the electric signal of any one range of a plurality of visual field ranges set along a guiding direction of the treatment instrument guiding member with respect to the light-receiving surface from the electric signal, a signal processing section that images the extracted visual field range corresponding to the electric signal extracted by the signal extraction section, a display section that displays an image of the extracted visual field range imaged by the signal processing section, an instruction section that instructs switching among the plurality of visual field ranges, a storage section that stores plurality of visual field ranges, and a control section that instructs, upon receiving an input from the instruction section, the signal extraction section to perform an extraction operation based on the extracted visual field range read from the storage section, instructs the signal processing section to image the extracted visual field range, controls the display section so as to show an enlarged view of the image of the extracted observation visual field with respect to the extracted visual field range of the objective optical system, and instructs the signal extraction section to perform an extraction operation so that every time there is an input from the instruction section, the image displayed on the display section is switched to another visual field range which is different from the current visual field range among the plurality of visual field ranges.

An endoscope system according to yet another aspect of the present invention includes an image pickup device that has a light-receiving surface on which an image of a site to be observed in a subject is formed via an objective optical system, a treatment instrument guiding member that guides a treatment instrument in an observation visual field of the objective optical system to the site to be observed in the subject, a signal extraction section that receives from the image pickup device, an electric signal of an entirety of the observation visual field of the objective optical system whose image is formed on the light-receiving surface and extracts the electric signal of any one range of a plurality of visual field ranges set along a guiding direction of the treatment instrument guiding member with respect to the light-receiving surface from the electric signal, a signal processing section that images the extracted visual field range corresponding to the electric signal extracted by the signal extraction section, a display section that displays an image of the extracted visual field range imaged by the signal processing section, a storage section that stores plurality of visual field ranges, and a control section that instructs the signal extraction section to perform an extraction operation based on the extracted visual field range read from the storage section, instructs the signal processing section to image the extracted visual field range, controls the display section so as to show an enlarged view of the image of the extracted observation visual field with respect to the extracted visual field range of the objective optical system, and instructs the signal extraction section to perform an extraction operation so that according to at least one of an operation state of the treatment instrument guiding member and a position of the treatment instrument guided by the treatment instrument guiding member, the image displayed on the display section is switched to another visual field range which is different from the current visual field range among the plurality of visual field ranges.

### BRIEF DESCRIPTION OF THE DRAWINGS

FIG. 1 is a perspective view schematically illustrating an example of a configuration of an endoscope system provided with a side-view type endoscope illustrating a first embodiment;

FIG. 2 is a partial perspective view of a distal end side of the insertion portion in FIG. 1 enlarged from a II direction in FIG. 1;

FIG. 3 is a partial cross-sectional view of the distal end portion along line III-III in FIG. 2;

FIG. 4 is a partial cross-sectional view of the distal end portion along line IV-IV in FIG. 2;

FIG. 5 is a block diagram schematically illustrating a configuration of an electric circuit that causes the monitor in FIG. 1 to display a site to be observed;

FIG. 6 is a diagram illustrating an observation visual field of the objective lens on the light-receiving surface in FIG. 5 and two visual field ranges set in the observation visual field;

FIG. 7 is a diagram illustrating a treatment instrument and a direction of visual field displayed together with a site to be observed on the monitor in FIG. 5;

FIG. 8 is a flowchart illustrating display control on the monitor by the control section in FIG. 5;

FIG. 9 is a diagram illustrating a modification of respective visual field range setting positions on the light-receiving surface in FIG. 6 together with an observation visual field of the objective lens;

FIG. 10 is a diagram illustrating a modification in which two or more visual field ranges set within the observation visual field are set on the light-receiving surface in FIG. 6 together with the observation visual field of the objective lens;

FIG. 11 is a diagram illustrating a modification in which two visual field ranges set within the observation visual field are set on the light-receiving surface in FIG. 6 in a tilt direction of the light-receiving surface of the image pickup device together with the observation visual field of the objective lens;

FIG. 12 is a diagram illustrating a modification in which a plurality of visual field ranges are set in all observation visual fields on the light-receiving surface in FIG. 6 together with the observation visual field of the objective lens;

FIG. 13 is a diagram illustrating an observation visual field of an objective lens, an image of which is formed on a light-receiving surface of an image pickup device in an endoscope system provided with a side-view type endoscope illustrating a second embodiment, two visual field ranges set within the observation visual field and a borderline between the two visual field ranges; and

FIG. 14 is a diagram illustrating a distribution of luminance in a tilt direction of the light-receiving section on the borderline of the light-receiving section in FIG. 13.

### DETAILED DESCRIPTION OF THE PREFERRED EMBODIMENTS

Hereinafter, embodiments of the present invention will be described with reference to the accompanying drawings. Note that a side-view type endoscope system will be taken as an example of an endoscope system in embodiments shown below.

### First Embodiment

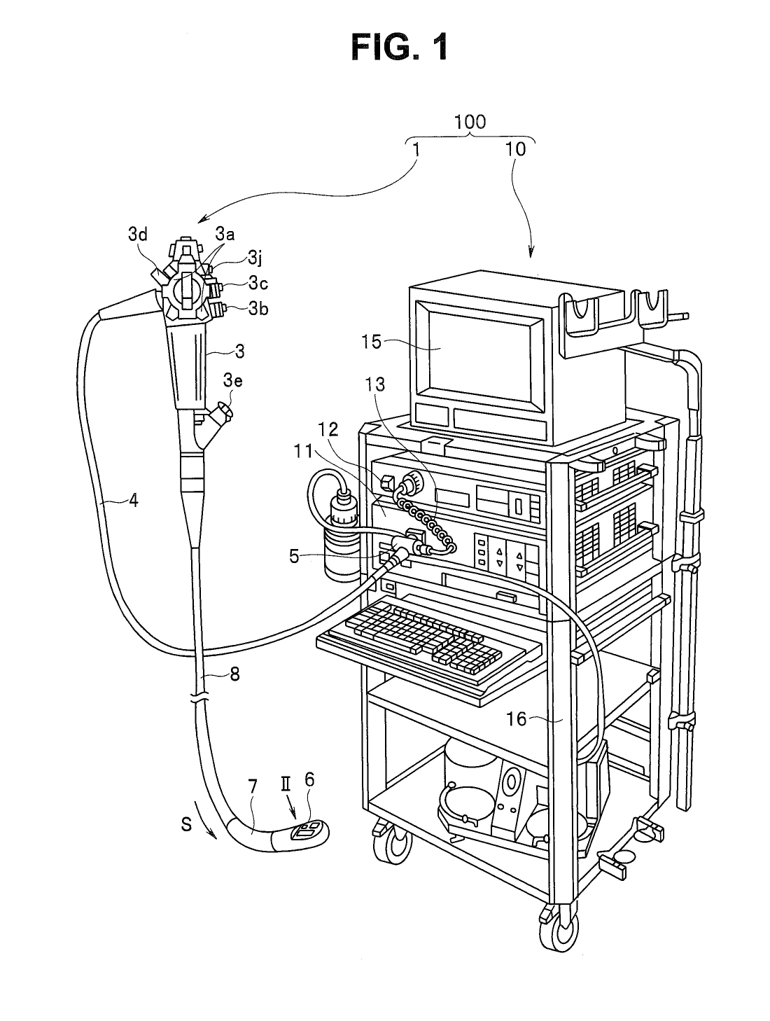

FIG. 1 is a perspective view schematically illustrating an example of a configuration of an endoscope system provided with a side-view type endoscope illustrating a first embodiment.

As shown in FIG. 1, a side-view type endoscope system 100 is constructed of an endoscope 1 and a peripheral apparatus 10.

The endoscope 1 is provided with an insertion portion 2 which is inserted into a subject and an operation section 3 connected to a proximal end of the insertion portion 2 in an insertion direction S (hereinafter simply referred to as a “proximal end”). The endoscope 1 is also provided with a universal cord 4 extending from the operation section 3 and a connector 5 provided at an extending end of the universal cord 4.

The peripheral apparatus 10 is provided, mounted on a stand 16, with a light source apparatus 11, a video processor 12, a connection cable 13 that electrically connects the light source apparatus 11 and the video processor 12 and a monitor 15 which is a display section.

The endoscope 1 and the peripheral apparatus 10 having such configurations are freely mutually connectable by the connector 5. The connector 5 is freely connectable to the light source apparatus 11, for example.

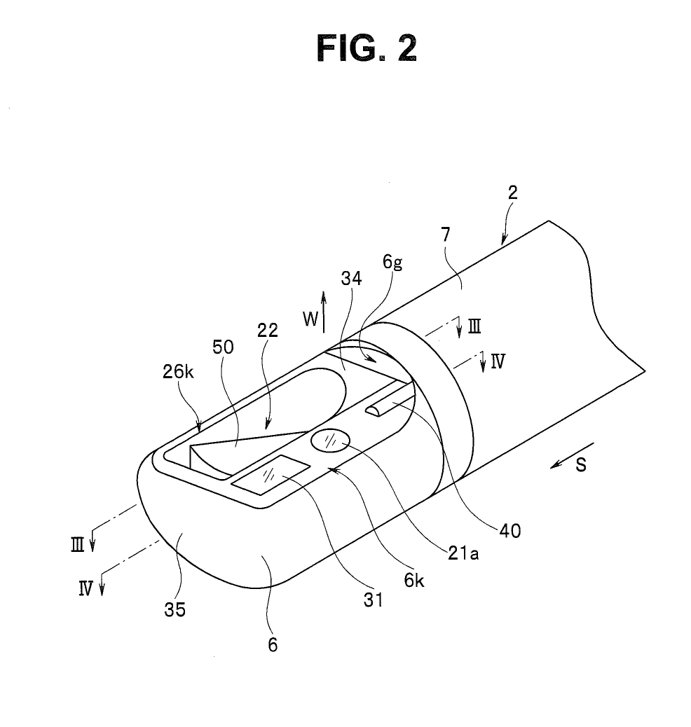

The operation section 3 is provided with two bending operation knobs 3a, an air/water feeding operation button 3b, a suction operation button 3c, and a treatment instrument base operation knob 3d that is rotated so as to raise or lower a treatment instrument raising base 50 which will be described later (see FIG. 2).

The operation section 3 is provided with a treatment instrument insertion port 3e to insert a treatment instrument 59 which will be described later (see FIG. 7) into a treatment instrument insertion channel 26 provided along the insertion direction S in the insertion portion 2 of the endoscope 1.

The operation section 3 is further provided with an instruction section 3j made up of a push button or the like for instructing switching between visual field ranges which will be described later of a site to be observed inside a subject displayed on the monitor 15.

Note that without being limited to the operation section 3, the instruction section 3j may be constructed of a button or the like in the video processor 12 or constructed of a keyboard or mouse or the like connected to the video processor 12. Furthermore, the instruction section 3j may also be constructed of a touch panel or the like provided on a screen of the monitor 15.

The insertion portion 2 is constructed of a distal end portion 6 located on a distal end side of the insertion portion 2 in the insertion direction S (hereinafter simply referred to as “distal end side”), a bending portion 7 connected to the proximal end of the distal end portion 6 and a flexible tube 8 connected to the proximal end of the bending portion 7.

The bending portion 7 is operated to be bent in four directions, up/down, left/right by the two bending operation knobs 3a provided in the operation section 3, for example.

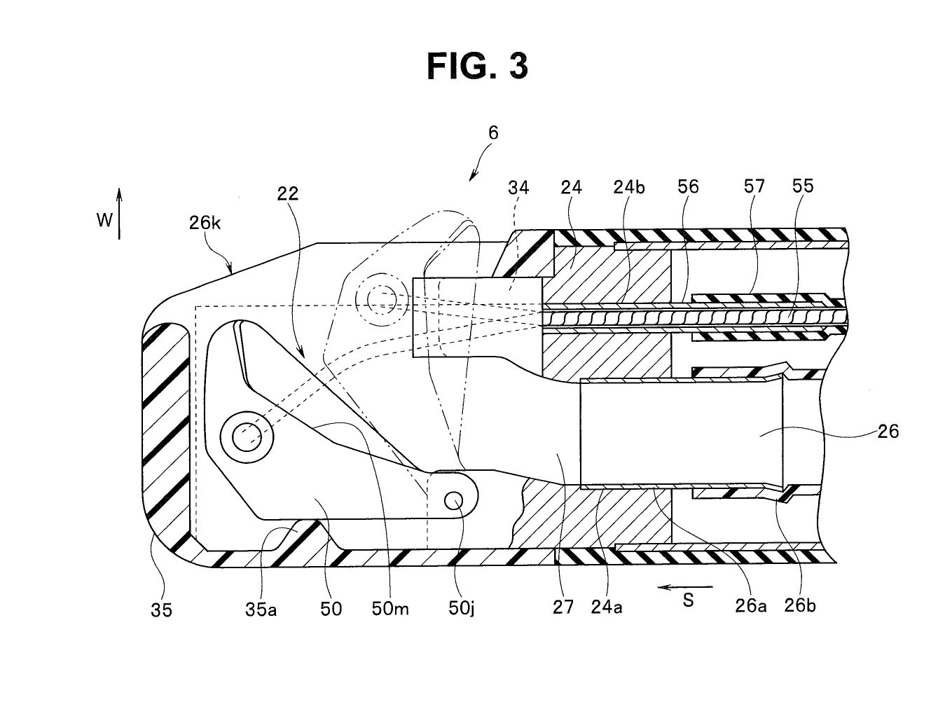

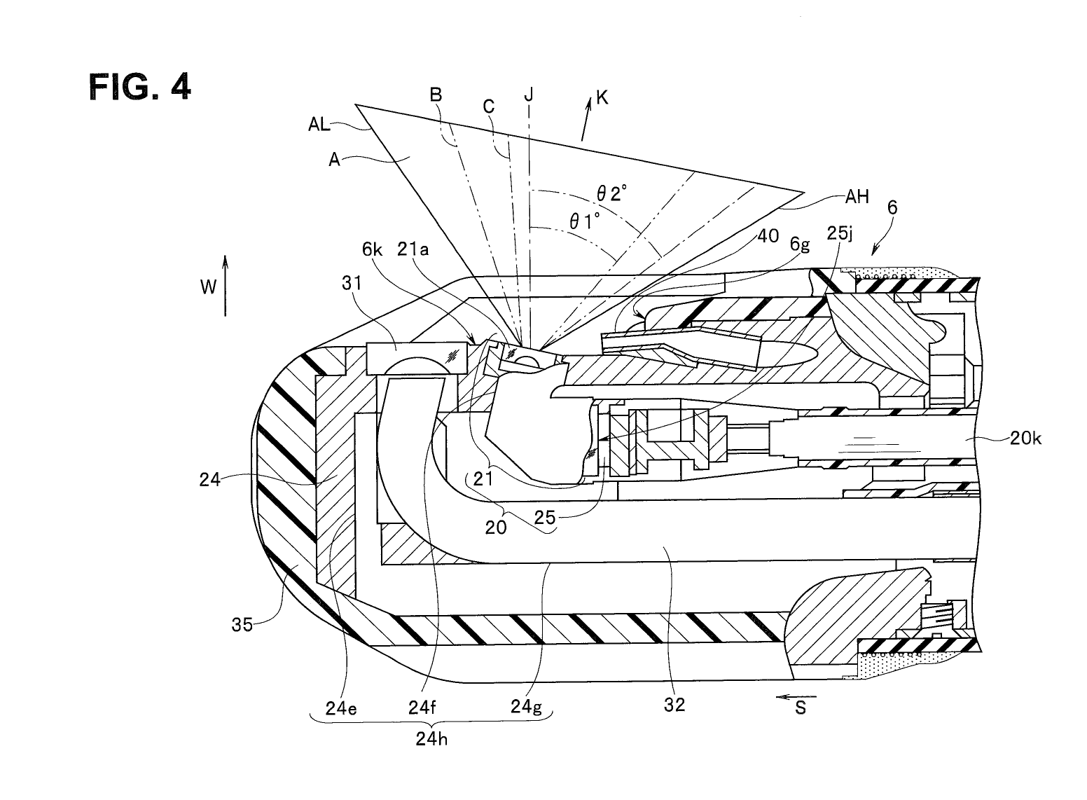

Next, a configuration of the distal end portion 6 of the insertion portion 2 in FIG. 1 will be described using FIG. 2 to FIG. 4. FIG. 2 is a partial perspective view of a distal end side of the insertion portion in FIG. 1 enlarged from a II direction in FIG. 1, FIG. 3 is a partial cross-sectional view of the distal end portion along line III-III in FIG. 2, and FIG. 4 is a partial cross-sectional view of the distal end portion along line IV-IV in FIG. 2.

As shown in FIG. 2, a notched portion 6k which is a concave portion with one outer circumferential face side of a distal end rigid portion 24 making up the distal end portion 6 notched out is formed in an outer circumferential face of the distal end portion 6. A channel opening 26k which is an opening of the distal end portion 6 of a treatment instrument insertion channel 26 (see FIG. 3) is provided on one outer circumferential face of this notched portion 6k.

Furthermore, an objective lens 21a which is an objective optical system in an image pickup unit 20 provided in the distal end portion 6 and an illumination lens 31 which is an illumination optical system are provided on the one outer circumferential face of the notched portion 6k and in the vicinity of the channel opening 26k as shown in FIG. 4.

Furthermore, an air/water feeding nozzle 40 is provided so as to protrude from a wall surface 6g formed of the notched portion 6k on the proximal end side of the notched portion 6k of the distal end portion 6 in the insertion direction S (hereinafter simply referred to as “proximal end side”).

This nozzle 40 is intended to clean an outer surface of the objective lens 21a by spraying a fluid such as water or air onto the outer surface of the objective lens 21a through a button operation of the air/water feeding operation button 3b of the operation section 3.

As shown in FIG. 3 and FIG. 4, the distal end portion 6 is provided with a distal end rigid portion 24 made of metal, for example, and a distal end cover 35 that covers a circumference of the distal end rigid portion 24 except the channel opening 26k, the objective lens 21a and the illumination lens 31. Note that the distal end cover 35 is made of a non-conductive material, for example, and fixed to the distal end rigid portion 24 using an adhesive or the like.

As shown in FIG. 3, a long hole 24a is formed in the distal end rigid portion 24 along the insertion direction S and an outer circumference of a connection pipe 26a on a distal end side is fitted in the long hole 24a. A distal end side of a tube 26b making up the treatment instrument insertion channel 26 is internally fixed to an outer circumference on a proximal end side of the connection pipe 26a.

Since the interior of the connection pipe 26a communicates with the interior of the tube 26b the interior of the connection pipe 26a also constitutes the treatment instrument insertion channel 26.

Note that the tube 26b is inserted through the insertion portion 2, the operation section 3, the universal cord 4 and the connector 5, and constitutes a suction duct. The suction duct is a duct that suctions a foreign substance or liquid or the like inside the subject through a button operation of the suction operation button 3c. Furthermore, as described above, the tube 26b located inside the operation section 3 is partially branched and opened to the treatment instrument insertion port 3e.

Furthermore, the opening at the distal end of the connection pipe 26a communicates with a space 22 formed of the distal end rigid portion 24 and the distal end cover 35 inside the distal end portion 6. Thus, the space 22 also constitutes the treatment instrument insertion channel 26.

A treatment instrument raising base 50 is provided in the vicinity of the channel opening 26k in the space 22 in the distal end portion 6, as a treatment instrument guiding member that changes an advancing direction of a treatment instrument 59 inserted from the treatment instrument insertion port 3e into the treatment instrument insertion channel 26 from the insertion direction S to the channel opening 26k side. The treatment instrument raising base 50 guides the treatment instrument 59 to a site to be observed in the subject along with elevation while changing the raising angle within an observation visual field A (see FIG. 4) of the objective lens 21a.

A guiding surface 50m for guiding the treatment instrument 59 to the channel opening 26k is formed on a surface of the treatment instrument raising base 50 opposite to the channel opening 26k and a groove or the like is formed in the guiding surface 50m so that the treatment instrument 59 can be freely fitted therein.

Note that the treatment instrument raising base 50 changes the advancing direction of not only the treatment instrument 59 but also a known guide wire that guides the treatment instrument 59 to a site to be observed in the subject.

A turning operation of the treatment instrument raising base operation knob 3d causes the treatment instrument raising base 50 to freely elevate or lower as shown by a solid line and one-dot dashed line in FIG. 3 by pulling or loosening, along the insertion direction S, of a wire 55 inserted through the insertion portion 2 and the operation section 3 and whose distal end is fixed on the distal end side of the treatment instrument raising base 50.

Note that a distal end side of a guide pipe 56 is fitted into a long hole 24b formed in the distal end rigid portion 24 along the insertion direction S. The wire 55 is inserted through the guide pipe 56 and the wire 55 thereby penetrates the long hole 24b along the insertion direction S. A distal end side of a guide tube 57 that coats a circumference of the wire 55 inserted into the insertion portion 2 is coated and fixed to a circumference of the guide pipe 56 on the proximal end side.

The treatment instrument raising base 50 has a substantially triangular cross section and can freely elevate or lower since the distal end side thereof is freely pivotable around a rotation shaft 50j on the proximal end side fixed to the distal end rigid portion 24 in the space 22 caused by pulling or loosening of the wire 55 fixed to the distal end side.

Note that as shown by one-dot dashed line in FIG. 3, a maximum raised position of the treatment instrument raising base 50 is defined by the treatment instrument raising base 50 contacting a receiving member 34 made of an insulating member located closer to the distal end side than the aforementioned wall surface 6g of the distal end portion 6 and provided at the distal end rigid portion 24. A maximum lowered position of the treatment instrument raising base 50 is defined by a rear surface of the treatment instrument raising base 50 contacting a convex portion 35a provided on the distal end cover 35.

Note that the treatment instrument raising base 50 and the treatment instrument 59 raised by the treatment instrument raising base 50 are displayed on the monitor 15 as moving from bottom to top of the screen along with elevation.

As shown in FIG. 4, an image pickup unit arrangement hole 24h in which the image pickup unit 20 is mounted is formed in the distal end rigid portion 24.

More specifically, the image pickup unit arrangement hole 24h is provided with a space 24g formed along the insertion direction S with respect to the distal end rigid portion 24. The image pickup unit arrangement hole 24h is also provided with a space 24f opened in the notched portion 6k of the distal end portion 6 formed so as to cross the distal end side of the space 24g along a direction K inclined rearward by a set angle from an axis J crossing the insertion direction S at 90°.

The image pickup unit 20 is provided in the spaces 24f and 24g in such a way that the objective lens 21a faces the notched portion 6k. Main parts of the image pickup unit 20 are configured by including a plurality of lenses 21 including the objective lens 21a and an image pickup device 25 including a light-receiving surface 25j on which an image of a site to be observed in the subject is formed via the plurality of lenses 21. Note that the image pickup device 25 may also be provided in the operation section 3.

That is, the endoscope 1 of the present embodiment is a side-view type endoscope with the objective lens 21a observing sideways W with respect to the insertion direction S. Furthermore, in the present embodiment, the objective lens 21a is made up of a super-wide-angle lens with a view angle of an observation visual field A shown by a solid line in FIG. 4 being 170°, for example.

Note that a signal cable 20k that electrically connects the image pickup unit 20 and the video processor 12 (see FIG. 1) extends from the image pickup unit 20. The signal cable 20k is inserted through the insertion portion 2, the operation section 3 and the universal cord 4, and electrically connected to the video processor 12 via the connector 5, the light source apparatus 11 and the connection cable 13.

In this way, an image of the site to be observed in the subject picked up by the image pickup unit 20 is transmitted to the video processor 12 via the signal cable 20k, subjected to image processing in the video processor 12 and then displayed on the monitor 15.

Note that the treatment instrument raising base 50 and the treatment instrument 59 raised by the treatment instrument raising base 50 are displayed along with elevation on the light-receiving surface 25j as in the case of the monitor 15 as moving from bottom to top of the light-receiving surface 25j, that is, from a lower side AL to an upper side AH of an observation visual field A. Here, the lower side AL of the observation visual field A corresponds to an observation limit on the front side in the observation visual field A and the upper side AH corresponds to an observation limit on the rear side in the observation visual field A as shown in FIG. 4.

Furthermore, the image pickup unit arrangement hole 24h is also provided with a space 24e opened in the notched portion 6k, formed so as to cross the space 24g at a position closer to the distal end side than the position at which the space 24f crosses the space 24g.

Moreover, a light guide 32 is inserted closer to the bottom side (lower side in FIG. 4) than the image pickup unit 20 in the space 24g. Note that the light guide 32 is inserted through the insertion portion 2, the operation section 3 and the universal cord 4, and has a configuration in which light supplied from the light source apparatus 11 (see FIG. 1) impinges on the proximal end thereof at the connector 5.

The distal end side of the light guide 32 inserted through the space 24g is provided, after being inserted through the space 24e, at a position opposite to the illumination lens 31 provided so as to face the opening in the notched portion 6k of the space 24e.

In this way, light emitted from the light source apparatus 11 is guided through the light guide 32 from the proximal end to the distal end and radiated from the illumination lens 31 into the subject.

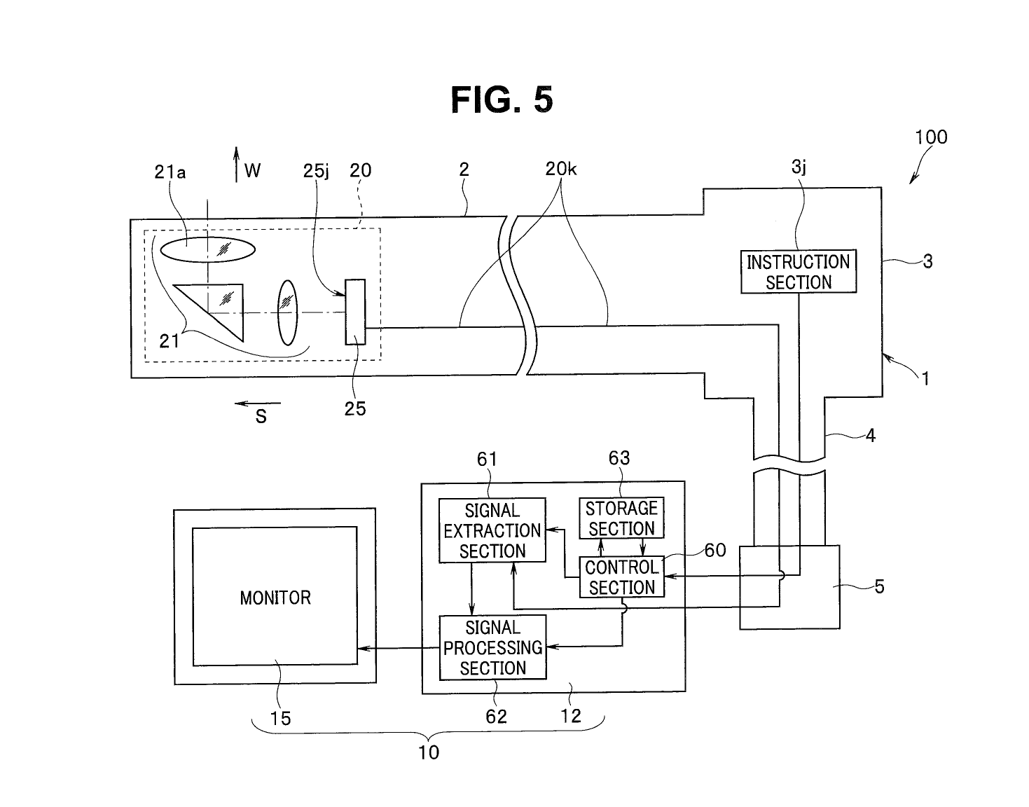

Next, a configuration of an electric circuit that displays an image of the site to be observed in the subject picked up by the image pickup unit on the display section will be described using FIG. 5 to FIG. 7.

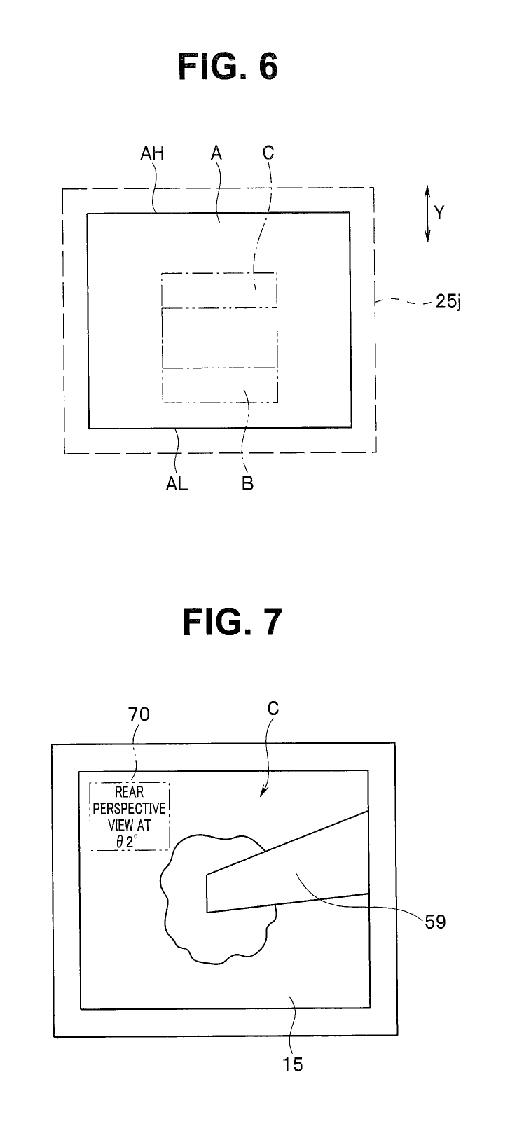

FIG. 5 is a block diagram schematically illustrating a configuration of an electric circuit that causes the monitor in FIG. 1 to display a site to be observed, FIG. 6 is a diagram illustrating an observation visual field of the objective lens on the light-receiving surface in FIG. 5 and two visual field ranges set in the observation visual field and FIG. 7 is a diagram illustrating a treatment instrument and a direction of visual field displayed together with a site to be observed on the monitor in FIG. 5.

As shown in FIG. 5, the video processor 12 incorporates a control section 60, a signal extraction section 61, a signal processing section 62 and a storage section 63.

The signal extraction section 61 is electrically connected to the image pickup device 25 via the signal cable 20k.

The signal extraction section 61 receives an electric signal of the entire observation visual field A shown in FIG. 4 in the objective lens 21a, an image of which is formed on the light-receiving surface 25j of the image pickup device 25 via the signal cable 20k from the image pickup device 25 all the time during which the image pickup device 25 is operating.

Furthermore, the signal extraction section 61 is also provided with a function of extracting from the transmitted electric signal, an electric signal within one of a plurality of visual field ranges B and C smaller than the observation visual field A and set along a lifting direction Y which is the guiding direction of the treatment instrument raising base 50 for the light-receiving surface 25j as shown in FIG. 6. Note that the signal extraction section 61 may also be provided in the endoscope 1.

Here, the visual field range B is set so as to have a view angle of 100°, for example, as shown by a two-dot dashed line in FIG. 4 and FIG. 6 within the observation visual field A of the objective lens 21a shown by a solid line in FIG. 4 and set to within a range having a rearview angle of θ1° behind the axis J that crosses the insertion direction S at 90°.

On the other hand, the visual field range C is set so as to have a view angle of 100°, for example, as shown by a one-dot dashed line in FIG. 4 and FIG. 6 within the observation visual field A of the objective lens 21a shown by the solid line in FIG. 4 and set to within a range having a rearview angle of θ2° which is greater than θ1° behind the axis J that crosses the insertion direction S at 90°.

Note that the view angles 100° of the visual field range B and the visual field range C are substantially equal to a view angle of an objective lens of a normal side-view type endoscope. That is, a view angle 170° of the observation visual field A of the objective lens 21a according to the present embodiment is set to an angle considerably greater than the view angle of the objective lens of a normal side-view type endoscope.

Moreover, the visual field range C is set to be adjacent to and above the visual field range B in FIG. 6 in the lifting direction Y. This is because the rearview angle θ2° of the visual field range C is greater than the rearview angle θ1° of the visual field range B as described above.

The visual field range C is set to be adjacent to the lifting direction Y so as to partially overlap with the visual field range B. Note that in the present embodiment, the visual field range C is set to the same size as the visual field range B, but may be set to a different size.

The signal processing section 62 is electrically connected to the signal extraction section 61 and has a function of imaging one of the visual field ranges B and C corresponding to an electric signal extracted by the signal extraction section 61. Note that the signal processing section 62 may also be provided inside the endoscope 1.

The signal processing section 62 is electrically connected to the monitor 15 and an image of one of the extracted visual field ranges B and C which has been imaged by the signal processing section 62 is displayed on the monitor 15.

Note that no image of the observation visual field A is displayed on the monitor 15. This is because if an image of the observation visual field A having a view angle of 170° is displayed on the monitor 15, images displayed on the monitor 15 may have a smaller magnification as described above.

Moreover, the image of one of the visual field ranges B and C displayed on the monitor 15 is displayed in a magnified view with respect to the observation visual field A. Thus, a region desired by an observer in the site to be observed is displayed magnified. Suppose the storage section 63 stores the visual field ranges B and C.

The control section 60 is electrically connected to the instruction section 3j, the signal extraction section 61, the signal processing section 62 and the storage section 63, receives an input from the instruction section 3j that instructs switching between the visual field ranges and instructs the signal extraction section 61 to perform an extraction operation based on one of the visual field ranges B and C read from the storage section 63.

The control section 60 instructs the signal processing section 62 to image one of the extracted visual field ranges B and C and controls the monitor 15 to show an image of one of the extracted visual field ranges B and C magnified with respect to the observation visual field A.

Furthermore, the control section 60 instructs the signal extraction section 61 to perform an extraction operation so that an image displayed on the monitor 15 is switched over to an image of another visual field range different from the current visual field range of the plurality of visual field ranges B and C every time there is an input from the instruction section 3j.

More specifically, every time there is an input from the instruction section 3j, the control section 60 instructs the signal extraction section 61 to perform an extraction operation so that an image displayed on the monitor 15 is switched over to an image of another visual field range adjacent to the current visual field range in the lifting direction Y of the plurality of visual field ranges B and C.

Furthermore, the control section 60 controls the monitor 15 so as to display one of the visual field ranges B and C as shown in FIG. 7, for example and display a direction of visual field 70 of the visual field range extracted by the signal extraction section 61.

More specifically, as shown in FIG. 7, when the control section 60 controls the monitor 15 so as to display the visual field range C, for example, the control section 60 performs control so as to display characters “rear perspective view at θ2°” corresponding to the visual field range C. Note that the direction of visual field 70 is not limited to characters, but may be a display of only a symbol such as an arrow or numeric characters.

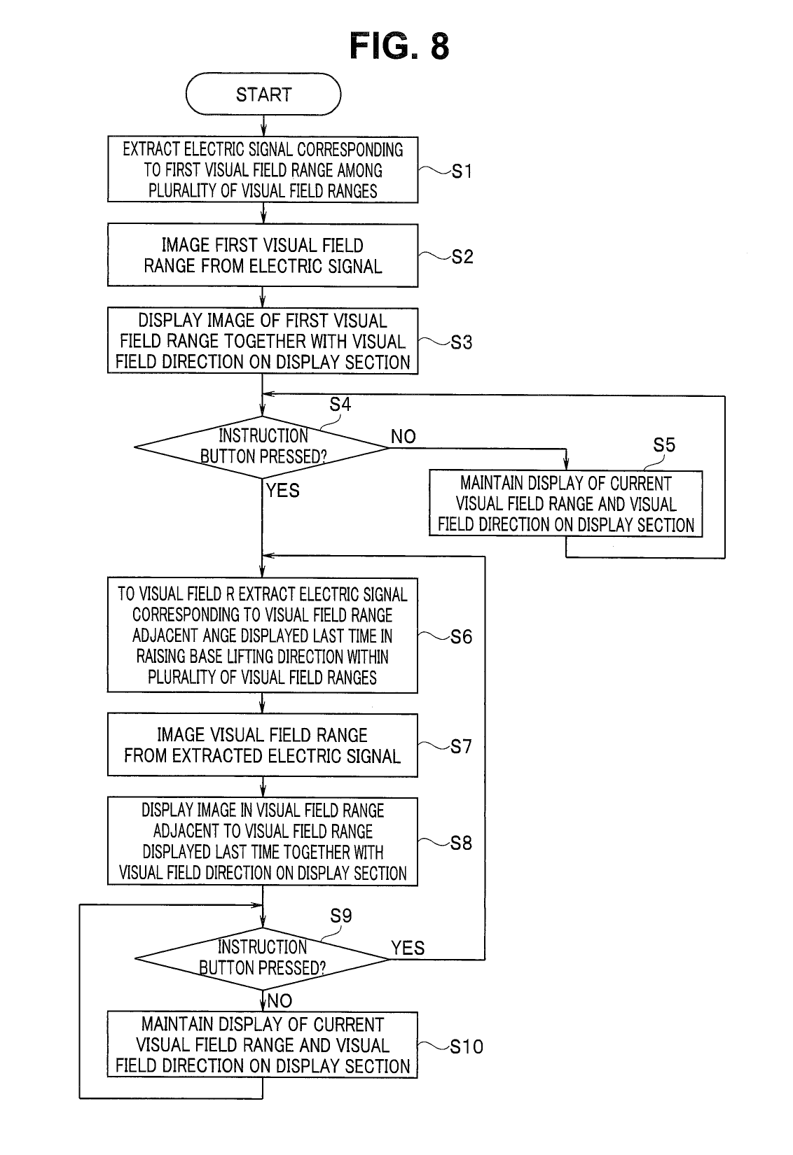

Next, a display of one of the visual field ranges B and C on the monitor 15 by the control section 60 in FIG. 5 and display control of the direction of visual field 70 will be described using a flowchart in FIG. 8. FIG. 8 is a flowchart illustrating display control on the monitor by the control section in FIG. 5.

As shown in FIG. 8, first in step S1, when an electric signal of the entire observation visual field A shown in FIG. 4 in the objective lens 21a, an image of which is formed on the light-receiving surface 25j of the image pickup device 25, is transmitted to the signal extraction section 61 from the image pickup device 25 via the signal cable 20k, the control section 60 instructs the signal extraction section 61 to extract an electric signal corresponding to a first visual field range, for example, the visual field range B from among the plurality of visual field ranges B and C read from the storage section 63.

Next, in step S2, the control section 60 instructs the signal processing section 62 to image the extracted visual field range B.

After that, in step S3, the control section 60 controls the monitor 15 to display an image of the visual field range B together with the direction of visual field 70 so that the treatment instrument 59 is located in the center of the monitor 15. More specifically, the control section 60 controls the monitor 15 to display the image of the visual field range B magnified with respect to the observation visual field A and characters “rear perspective view at θ1°” and proceeds to step S4.

In step S4, the control section 60 determines whether or not the instruction button making up the instruction section 3j is pressed, that is, whether or not there is an input from the instruction section 3j. If the instruction button is not pressed, that is, there is no input from the instruction section 3j, the flow branches to step S5, where the control section 60 controls the monitor 15 to continue to display the current visual field range B and the direction of visual field 70. Then, step S4 and step S5 are repeated until the instruction button is pressed.

When the instruction button is pressed in step S4, that is, there is an input from the instruction section 3j, in step S6, the control section 60 instructs the signal extraction section 61 to extract an electric signal corresponding to a visual field range different from the visual field range B displayed last time, that is, an electric signal corresponding to the visual field range C adjacent to in the lifting direction Y and partially overlapping with the visual field range B.

Next, in step S7, the control section 60 instructs the signal processing section 62 to image the extracted visual field range C.

After that, in step S8, as shown in FIG. 7, the control section 60 controls the monitor 15 to display an image of the visual field range C together with the direction of visual field 70 such that the treatment instrument 59 is located in the center of the monitor 15. More specifically, the control section 60 controls the monitor 15 to display the image of the visual field range C magnified with respect to the observation visual field A and characters “rear perspective view at θ2°” and proceeds to step S9.

In step S9, the control section 60 determines whether or not the instruction button making up the instruction section 3j is pressed, that is, whether or not there is an input from the instruction section 3j.

If the instruction button is not pressed, that is, there is no input from the instruction section 3j, the flow branches to step S10, where the control section 60 controls the monitor 15 to continue to display the current visual field range C and the direction of visual field 70, and repeats step S9 and step S10 until the instruction button is pressed.

In step S9, if the instruction button is pressed, that is, if there is an input from the instruction section 3j, the control section 60 returns to step S6, instructs the signal extraction section 61 to extract an electric signal corresponding to a visual field range different from the visual field range C displayed last time, that is, an electric signal corresponding to the visual field range B adjacent to the visual field range C in the lifting direction Y, and then repeats step S6 to step S10.

That is, in step S6, every time there is an input from the instruction section 3j, the control section 60 instructs the signal extraction section 61 to perform an extraction operation so that an image displayed on the monitor 15 is switched over to an image of another visual field range adjacent to the current visual field range in the lifting direction Y of the plurality of visual field ranges B and C.

Thus, in the present embodiment, the monitor 15 displays one of the visual field range B and the visual field range C having a view angle of 100°, for example.

The present embodiment has described that every time the instruction button making up the instruction section 3j is pressed, an image displayed on the monitor 15 is switched between the visual field range B and the visual field range C set to be adjacent to each other in the lifting direction Y within the observation visual field A having a super-wide view angle of 170°, for example.

The present embodiment has also described that the visual field range B and the visual field range C are displayed on the monitor 15 magnified with respect to the observation visual field A such that the treatment instrument 59 is located in the center of the monitor 15.

Thus, since the visual field range B and the visual field range C are displayed on the monitor 15 magnified with respect to the observation visual field A, the observer can acquire a desired enlarged image at a site to be observed from the image of the visual field range B or the image of the visual field range C.

Furthermore, as described above, the treatment instrument raising base 50 and the treatment instrument 59 raised by the treatment instrument raising base 50 are displayed on the monitor 15 as moving from bottom to top of the screen along with elevation. From this, for example, when inserting the treatment instrument 59 into the bile duct, as described above, for reasons related to the position at which the bile duct extends from the papilla, the greater the raising angle of the treatment instrument raising base 50, the easier it becomes to insert the treatment instrument 59 into the bile duct, and therefore when the observer switches over to the visual field range C to perform observation, the treatment instrument 59 and papilla are displayed in a magnified view as shown in FIG. 7. It is therefore easier to insert the treatment instrument 59 after elevation.

Furthermore, as described above, when raising the treatment instrument 59 with weak resistance, if the raising angle of the treatment instrument raising base 50 is excessively increased, the treatment instrument 59 may be broken. From this, the raising angle of the treatment instrument raising base 50 needs to be decreased, but in this case, if the observer switches over to the visual field range B to perform observation, the periphery of the treatment instrument 59 is displayed in a magnified view even when the raising angle of the treatment instrument raising base 50 is small For this reason, even in this case, it is easy to insert the treatment instrument 59 after raising.

The present embodiment also has described that the visual field range B and the visual field range C in the observation visual field A are set so as to be adjacent to each other in the lifting direction Y and both have a view angle of 100°, for example.

Thus, the visual field range B and the visual field range C have wide view angles respectively and are set along the lifting direction Y of the treatment instrument raising base. For this reason, even when the visual field range is switched over to the visual field range B or the visual field range C, the treatment instrument 59 raised by the treatment instrument raising base 50 is never overlooked.

Even while a site to be observed is being observed, by only pressing the instruction button making up the instruction section 3j provided in the operation section 3, it is possible to easily switch an image displayed on the monitor 15 between the visual field range B and the visual field range C.

Thus, an enlarged view of a desired region of a site to be observed is shown irrespective of whether the site to be observed is being observed or the raising operation by the treatment instrument raising base 50 is in progress, and therefore it is not necessary to set a center position of the observation visual field and set a desired visual field range using the operation member provided in the operation section, as in the case of the prior art.

Furthermore, the present embodiment has described that the monitor 15 displays the direction of visual field 70 together with the visual field range B or the visual field range C.

Based on this, when switching between the visual field ranges, the observer can easily recognize from the monitor 15 in which direction of visual field the switched visual field range is seen. From this, it is possible to improve insertability of the treatment instrument 59 and thereby prevent damage of the treatment instrument 59 due to excessive elevation of the treatment instrument raising base 50 or the like.

It is therefore possible to provide the side-view type endoscope system 100 provided with a configuration capable of changing a visual field range in the observation visual field A of the objective lens 21a through a simple operation and thereby displaying, in a magnified view, the periphery of the treatment instrument 59 raised or lowered by the treatment instrument raising base 50 within the changed visual field range.

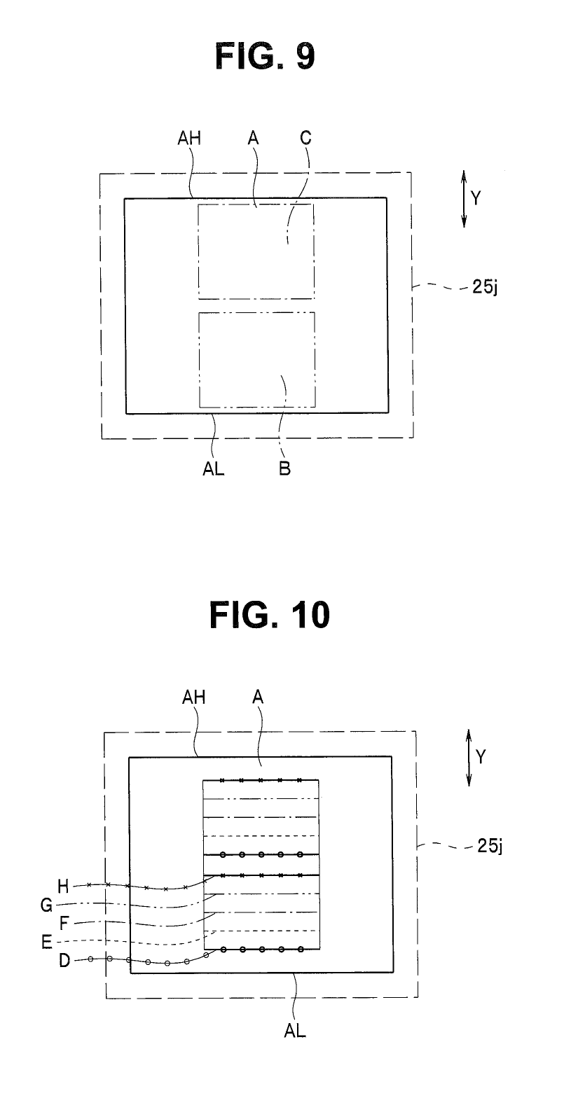

Note that hereinafter, a modification will be described using FIG. 9. FIG. 9 is a diagram illustrating a modification of respective visual field range setting positions on the light-receiving surface in FIG. 6 together with an observation visual field of the objective lens.

The aforementioned present embodiment has described that as shown in FIG. 6, among the plurality of visual field ranges B and C which are smaller than the observation visual field A and set along the lifting direction Y of the treatment instrument raising base 50 in the observation visual field A, the visual field range C is set above the visual field range B in the lifting direction Y in FIG. 6 and set so as to be adjacent to the visual field range B in the lifting direction Y.

Not being limited to this, as shown in FIG. 9, the visual field range B may be provided close to the vicinity of a lower side AL of an observation visual field A in FIG. 9 within the observation visual field A. The visual field range C may be provided so as to be adjacent to the visual field range B in the lifting direction Y and close to the vicinity of an upper side AH of the observation visual field A in FIG. 9 within the observation visual field A.

The aforementioned present embodiment shown in FIG. 6 has described that the visual field range B and the visual field range C which are adjacent to each other in the lifting direction Y are set so as to partially overlap with each other. Not being limited to this, however, the visual field range B and the visual field range C may be separated apart without partially overlapping with each other and set so as to be adjacent to each other in the lifting direction Y as shown in FIG. 9.

Note that though not shown, the visual field range B and the visual field range C may be set so that the lower side of the visual field range C in FIG. 9 matches the upper side of the visual field range B in FIG. 9. That is, an arrangement of the visual field range B and the visual field range C so as to be adjacent to each other in the lifting direction Y may be made in any form; whether they are separate from each other, overlapping with each other or sides of the respective visual field ranges coinciding with each other.

Note that even with such a configuration, every time the instruction button making up the instruction section 3j provided in the operation section 3 is pressed, an image displayed on the monitor 15 is switched between the visual field range B and the visual field range C under the drive control of the control section 60 shown in FIG. 8. The rest of the configuration is the same as that of the aforementioned present embodiment.

Based on this, in addition to the effects of the aforementioned present embodiment, it is possible to set the visual field range B and the visual field range C without a break so as to be adjacent to each other in the lifting direction Y between the upper side AH of the observation visual field A and the lower side AL of the observation visual field A in the lifting direction Y. Thus, even when an image displayed on the monitor 15 is switched between the visual field range B and the visual field range C, the treatment instrument 59 is always displayed in the observation visual field A of the objective lens 21a, and therefore the treatment instrument 59 is never overlooked.

In addition, another modification will be described below using FIG. 10. FIG. 10 is a diagram illustrating a modification in which two or more visual field ranges set within the observation visual field are set on the light-receiving surface in FIG. 6 together with the observation visual field of the objective lens.

The aforementioned present embodiment has described as shown in FIG. 6 that there are two visual field ranges B and C which are visual field ranges set along the lifting direction Y in the observation visual field A.

Without being limited to this, as shown in FIG. 10, the number of visual field ranges is not limited to two if they are set along the lifting direction Y in the observation visual field A, and, for example, five visual field ranges may be set, for example, visual field ranges D, E, F, G and H, or in addition, a plurality of visual field ranges may be set.

Note that the visual field ranges D to H are also stored in the storage section 63. The visual field ranges D, E, F, G and H may be set in different sizes.

With such a configuration, when the instruction button making up the instruction section 3j provided in the operation section 3 is pressed, an image displayed on the monitor 15 is switched every time the instruction button is pressed, from the visual field range D to E, from E to F, from F to G, from G to H, or conversely, from H to G, from G to F, from F to E, and from E to D under the drive control of the control section 60 shown in FIG. 8. Note that every time the instruction button is pressed, a visual field range may be switched to another one which is not adjacent thereto. The rest of the configuration is the same as that of the aforementioned present embodiment.

According to such a configuration, in addition to the effects of the aforementioned present embodiment, it is possible to set a plurality of visual field ranges along the lifting direction Y within the observation visual field A. For this reason, it is possible to improve followability of the treatment instrument 59 raised by the treatment instrument raising base 50 and easily switch between visual field ranges by only pressing the instruction button at a position at which the treatment instrument 59 is reflected in the center of the monitor 15.

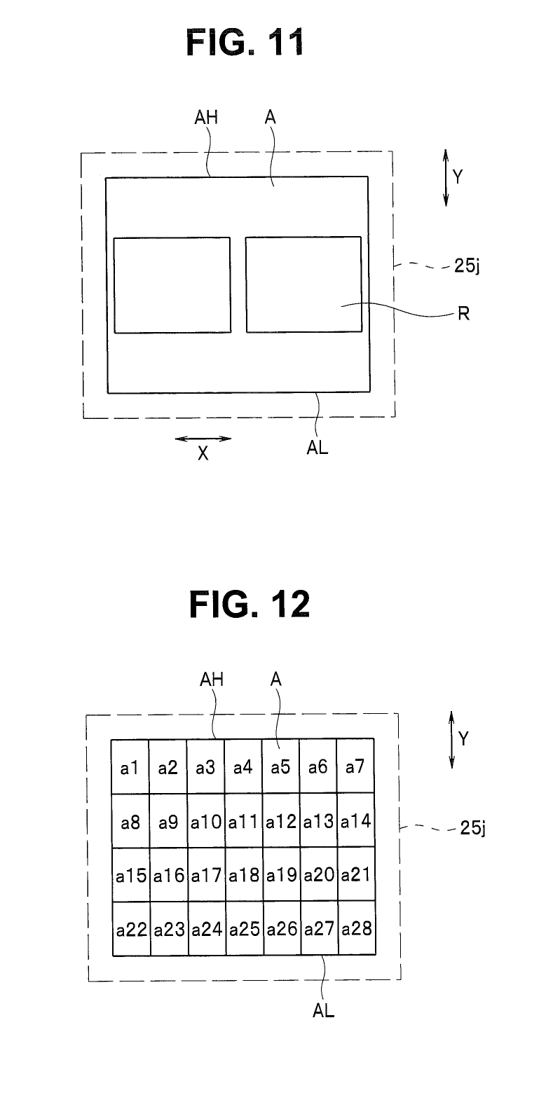

Yet another modification will be described below using FIG. 11. FIG. 11 is a diagram illustrating a modification in which two visual field ranges set within the observation visual field are set on the light-receiving surface in FIG. 6 in a tilt direction of the light-receiving surface of the image pickup device together with the observation visual field of the objective lens.

The aforementioned present embodiment has described as shown in FIG. 6 that there are two visual field ranges B and C which are visual field ranges set along the lifting direction Y in the observation visual field A.

Without being limited to this, as shown in FIG. 11, visual field ranges may be set as visual field ranges L and R adjacent to each other along a tilt direction X orthogonal to a lifting direction Y in the observation visual field A. Note that the visual field ranges L and R are also stored in the storage section 63.

In this case, the visual field ranges L and R may also be set so as to partially overlap with each other in the tilt direction X or set so that the right side of the visual field range L coincides with the left side of the visual field range R. Furthermore, the visual field range L may be set to a size different from that of the visual field range R. Note that the rest of the configuration is the same as that of the aforementioned present embodiment.

With such a configuration, every time the instruction button making up the instruction section 3j provided in the operation section 3 is pressed, an image displayed on the monitor 15 is switched between the visual field range L and the visual field range R under the drive control of the control section 60 shown in FIG. 8.

According to such a configuration, in addition to the effects of the aforementioned present embodiment, it is possible to switch an image displayed on the monitor 15 between the visual field range L and the visual field range R adjacent to each other in the tilt direction X. For this reason, it is possible to prevent such a case where the treatment instrument raising base 50 is displayed on the monitor 15 and the site to be observed and the treatment instrument 59 are made harder to see due to the presence of the treatment instrument raising base 50, by switching images between the visual field range L and the visual field range R to thereby hide the treatment instrument raising base 50 on the monitor 15.

Yet another modification will be described using FIG. 12. FIG. 12 is a diagram illustrating a modification in which a plurality of visual field ranges are set in all observation visual fields on the light-receiving surface in FIG. 6 together with the observation visual field of the objective lens.

As shown in FIG. 12, a plurality of visual field ranges may be set in the entire observation visual field A. More specifically, for example, 28 visual field ranges a1 to a28 may be set in the entire observation visual field A.

Note that the number of visual field ranges set the entire observation visual field A is not limited to 28. Moreover, the visual field ranges a1 to a28 are also stored in the storage section 63. The visual field ranges a1 to a28 may be set in different sizes. Note that the rest of the configuration is the same as that of the aforementioned present embodiment.

With such a configuration, when the instruction button making up the instruction section 3j provided in the operation section 3 is pressed, an image displayed on the monitor 15 is switched sequentially every time the instruction button is pressed, from the visual field ranges a1 to a28 under the drive control of the control section 60 shown in FIG. 8 or conversely switched sequentially from the visual field ranges a28 to a1 . Note that every time the instruction button is pressed, a visual field range may be switched to another one which is not adjacent thereto.

According to such a configuration, in addition to the effects of the aforementioned present embodiment, it is possible to set visual field ranges in the entire observation visual field A and sequentially display the visual field ranges on the monitor 15 along with pressing of the instruction button, and therefore easily show enlarged views of all images in the observation visual field A on the monitor 15.

### Second Embodiment

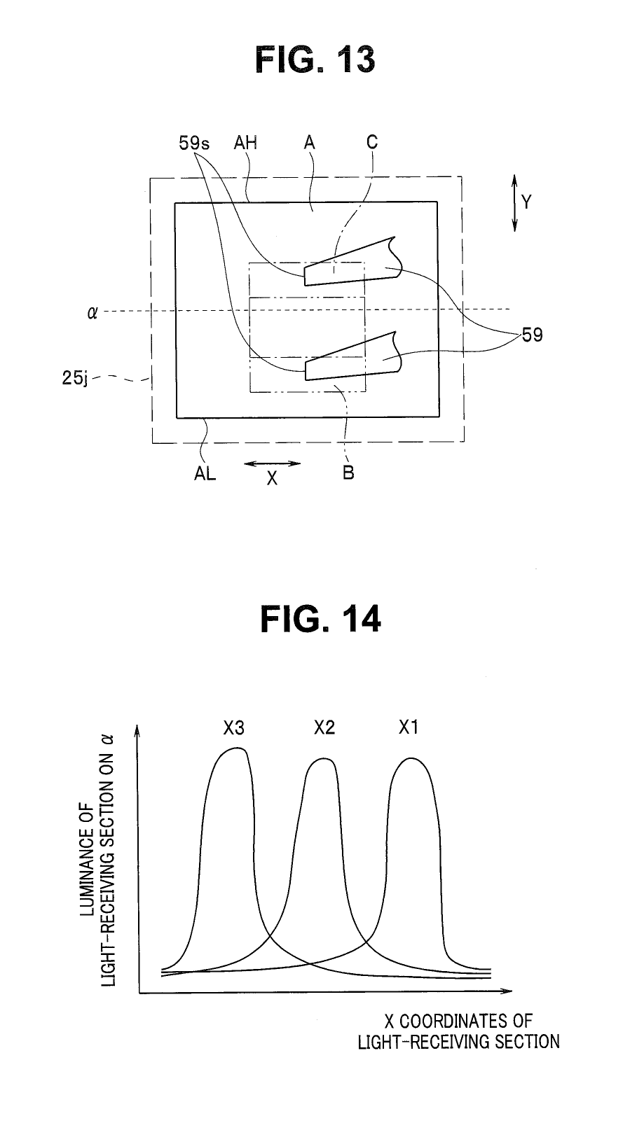

FIG. 13 is a diagram illustrating an observation visual field of an objective lens, an image of which is formed on a light-receiving surface of an image pickup device in an endoscope system provided with a side-view type endoscope illustrating the present embodiment, two visual field ranges set within the observation visual field and a borderline between the two visual field ranges and FIG. 14 is a diagram illustrating a distribution of luminance in a tilt direction of the light-receiving section on the borderline of the light-receiving section in FIG. 13.

The configuration of the side-view type endoscope system according to the second embodiment is different from the configuration of the side-view type endoscope system according to the first embodiment shown in aforementioned FIG. 1 to FIG. 8 in that it is further provided with a configuration in which a visual field range displayed on a monitor is automatically switched according to the raising position of a treatment instrument. Thus, components similar to those in the first embodiment are assigned the same reference numerals and description thereof will be omitted.

In the present embodiment, the control section 60 further performs control of instructing the signal extraction section 61 to perform an extraction operation so that an image displayed on the monitor 15 is automatically switched over to another visual field range which is different from the current visual field range among the visual field ranges B and C according to the lifting position of the treatment instrument 59 raised by the treatment instrument raising base 50.

More specifically, as shown in FIG. 13, a borderline a parallel to a tilt direction X is set in substantially the center of the lifting direction Y on the light-receiving surface 25j and the visual field ranges B and C are set within the observation visual field A on the light-receiving surface 25j as in the case of the aforementioned present embodiment in FIG. 6. The control section 60 has a function of reading an electric signal on the borderline a on the light-receiving surface 25j.

Here, as described above, in the present embodiment, the treatment instrument raising base 50 and the treatment instrument 59 raised by the treatment instrument raising base 50 are displayed on the light-receiving surface 25j as moving from bottom to top of the light-receiving surface 25j, that is, moving from a lower side AL to an upper side AH of the observation visual field A along with elevation.

Thus, when the treatment instrument 59 is raised using the treatment instrument raising base 50, the treatment instrument 59 is raised while keeping the tilt angle by the treatment instrument raising base 50 and illuminating light from the illumination lens 36 is radiated from below onto the treatment instrument 59. For this reason, the peak of brightness of the treatment instrument 59 moves from the root side of the treatment instrument 59 to the distal end 59s side on the borderline a as shown in FIG. 13. That is, as shown in FIG. 14, the peak moves from a peak X1 through a peak X2 to a peak X3. Note that the peak Xl to peak X3 are detected by the control section 60.

Thus, when the brightness peak changes from X1 through X2 to X3, the control section 60 determines that the treatment instrument 59 has passed through the borderline a from bottom to top and automatically switches the monitor 15 so as to display the visual field range C.

On the other hand, when the brightness peak changes from X3 through X2 to Xl, the control section 60 determines that the treatment instrument 59 has passed through the borderline a from top to bottom and automatically switches the monitor 15 so as to display the visual field range B.

Note that the above-described switching control by the control section 60 is likewise applicable to the case shown in FIG. 10 where two or more visual field ranges are set in the lifting direction Y. The rest of the configuration is the same as that of the aforementioned first embodiment.

According to such a configuration, along with elevation of the treatment instrument raising base 50, the visual field range is automatically switched according to the raising position of the treatment instrument 59 so that an enlarged image is displayed on the monitor 15 with the treatment instrument 59 being located in the center of the monitor 15 without the need for the observer to press the instruction button. From this, the observer can observe the enlarged image of the treatment instrument 59 from the monitor 15 without the need for any operation.

Note that the present embodiment is also provided with the configuration in which the visual field range is switched along with the pressing of the instruction button as in the case of the aforementioned first embodiment. From this, when it is not desirable to automatically switch the visual field range, it may be possible to avoid detection of luminance by the control section 60 and manually switch between visual field ranges as in the case of the aforementioned first embodiment.

Thus, the other effects are similar to those of the aforementioned first embodiment.

Automatic switching between visual field ranges is not limited to detection of luminance, but a colored portion may be provided at a distal end of the treatment instrument 59 and visual field ranges may be automatically switched every time the control section 60 detects the colored portion that passes through the borderline a along with elevation of the treatment instrument raising base 50.

Furthermore, the control section 60 may detect the angle of rotation of the treatment instrument raising base operation knob 3d, that is, the operation state of the treatment instrument raising base 50, the control section 60 may determine the raising angle of the treatment instrument 59 according to the angle of rotation and automatically switch between visual field ranges. That is, it goes without saying that it does not matter what configuration is adopted.

The above-described embodiment has described the invention by taking an endoscope system provided with a side-view type endoscope as an example, but it goes without saying that a similar invention is also applicable to an endoscope system provided with a front-view type endoscope.

In that case, a treatment instrument guiding member may be provided at the opening of the treatment instrument channel provided on a distal end face of the front-view type endoscope, which swings the treatment instrument that protrudes forward from this opening along a direction crossing the insertion direction.

In the case of a front-view type endoscope, it is often the case that the treatment instrument channel is set such that the treatment instrument protrudes from bottom to center of an observation screen, and causing the swinging direction of the treatment instrument by the treatment instrument guiding member to coincide with the horizontal direction on the observation screen improves the ease of use of the treatment instrument.

In such an endoscope, the moving direction of the aforementioned visual field range may also be caused to coincide with the horizontal direction in the observation visual field A.

What is claimed is:

1. An endoscope system comprising:
an image pickup device that has a light-receiving surface on which an image of a site to be observed in a subject is formed via an objective optical system;
a treatment instrument guiding member that guides a treatment instrument in an observation visual field of the objective optical system to the site to be observed in the subject;
a signal processing section that receives from the image pickup device, an electric signal of an entirety of the observation visual field whose image is formed on the light-receiving surface, extracts the electric signal of any one range of a plurality of visual field ranges set along a guiding direction of the treatment instrument guiding member with respect to the light-receiving surface from the electric signal and images the extracted visual field range corresponding to the electric signal; and
a display section that displays an image of the extracted visual field range imaged by the signal processing section.

2. An endoscope system comprising:
an image pickup device that has a light-receiving surface on which an image of a site to be observed in a subject is formed via an objective optical system;
a treatment instrument guiding member that guides a treatment instrument in an observation visual field of the objective optical system to the site to be observed in the subject;
a signal extraction section that receives from the image pickup device, an electric signal of an entirety of the observation visual field of the objective optical system whose image is formed on the light-receiving surface, and extracts the electric signal of any one range of a plurality of visual field ranges set along a guiding direction of the treatment instrument guiding member with respect to the light-receiving surface from the electric signal;
a signal processing section that images the extracted visual field range corresponding to the electric signal extracted by the signal extraction section;
a display section that displays an image of the extracted visual field range imaged by the signal processing section;
an instruction section that instructs switching among the plurality of visual field ranges;
a storage section that stores plurality of visual field ranges; and
a control section that instructs, upon receiving an input from the instruction section, the signal extraction section to perform an extraction operation based on the extracted visual field range read from the storage section, instructs the signal processing section to image the extracted visual field range, controls the display section so as to show an enlarged view of the image of the extracted observation visual field with respect to the extracted visual field range of the objective optical system, and instructs the signal extraction section to perform an extraction operation so that every time there is an input from the instruction section, the image displayed on the display section is switched to another visual field range which is different from the current visual field range among the plurality of visual field ranges.

3. The endoscope system according to claim 2, wherein every time there is an input from the instruction section, the control section instructs the signal extraction section to perform an extraction operation so that an image displayed on the display section is switched over to an image of another visual field range adjacent to the current visual field range in the guiding direction among the plurality of visual field ranges.

4. The endoscope system according to claim 2, wherein the control section causes the display section to further display a direction of visual field of the extracted visual field range.

5. The endoscope system according to claim 2, wherein at least one of each of the plurality of visual field ranges set is smaller than the observation visual field of the objective optical system.

6. The endoscope system according to claim 2, wherein an operation section is connected at a proximal end of the insertion portion in an insertion direction in the endoscope, and
the instruction section is provided in the operation section.

7. The endoscope system according to claim 2, wherein the control section further instructs the signal extraction section to perform an extraction operation so that the image displayed on the display section is switched over to another visual field range different from the current visual field range among the plurality of visual field ranges according to at least one of an operation state of the treatment instrument guiding member and a position of the treatment instrument guided by the treatment instrument guiding member.

8. An endoscope system comprising:
an image pickup device that has a light-receiving surface on which an image of a site to be observed in a subject is formed via an objective optical system;
a treatment instrument guiding member that guides a treatment instrument in an observation visual field of the objective optical system to the site to be observed in the subject;
a signal extraction section that receives from the image pickup device, an electric signal of an entirety of the observation visual field of the objective optical system whose image is formed on the light-receiving surface, and extracts the electric signal of any one range of a plurality of visual field ranges set along a guiding direction of the treatment instrument guiding member with respect to the light-receiving surface from the electric signal;
a signal processing section that images the extracted visual field range corresponding to the electric signal extracted by the signal extraction section;
a display section that displays an image of the extracted visual field range imaged by the signal processing section;
a storage section that stores the plurality of visual field ranges; and
a control section that instructs the signal extraction section to perform an extraction operation based on the extracted visual field range read from the storage section, instructs the signal processing section to image the extracted visual field range, controls the display section so as to show an enlarged view of the image of the extracted observation visual field with respect to the extracted visual field range of the objective optical system, and further instructs the signal extraction section to perform an extraction operation so that according to at least one of an operation state of the treatment instrument guiding member and a position of the treatment instrument guided by the treatment instrument guiding member, the image displayed on the display section is switched to another visual field range which is different from the current visual field range among the plurality of visual field ranges.
